(19) United States
(12) Patent Application Publication
(10) Pub. No.: US 2007/0118021 A1
(43) Pub. Date: May 24, 2007

(54) Anatomical cavity implant transport device and method

(76) Inventor: James L. Pokorney, Northfield, MN (US)

Correspondence Address: James L. Pokorney, 303 Washington Street, Northfield, MN, 55057, US

(21) Appl. No.: 11/603,588

(22) Filed: Nov. 21, 2006

Related U.S. Application Data
(60) Provisional application No. 60/739,359, filed on Nov. 22, 2005.

(51) Int. Cl. A61F 2/24; A61B 1/32

(52) U.S. Cl. 600/204; 623/2.11

(57) ABSTRACT

This invention describes a device and method for inserting a large diameter object into a blood filled, pressurized cavity in the body without applying undue trauma to the object as it passes through the device and without loosing significant amounts of blood. The invention comprises the following elements: three rigid hollow cylindrical elements aligned along a common axis, an elastomeric tubular element located within the housings, attachment means to connect the elastomeric tubular element to the three rigid hollow cylindrical elements, independent rotational means such as torsion springs to selectively bias the elastomeric tubular element into a twisted, closed configuration at each boundary between the three rigid hollow cylindrical elements, and a cavity access element to gain access into the anatomical cavity.

This application claims priority from provisional patent application U.S. Ser. No. 60/739,359 filed 2005 Nov. 22.

### BACKGROUND—FIELD OF INVENTION

This invention relates to an anatomical cavity implant transport device and associated method to insert relatively large implants or tools into a body cavity without loosing a significant amount of fluid contained within the cavity and without damaging the device during the insertion procedure. More specifically, this invention relates to a double valved device that allows the direct insertion of a large diameter heart valve prosthesis through the wall of a beating heart without significant blood loss or damage to the fragile valve.

### BACKGROUND—CLINICAL NEED

In many clinical applications, a doctor would like to insert a large implant or tool into a body cavity without loosing excessive fluid and without damaging the implant or tool during the insertion process. A good example of such an application is the insertion of a replacement prosthetic aortic heart valve via the apex of the left ventricle. To achieve this clinical goal, prior art designs represented by Andersen et al. in U.S. Pat. No. 6,168,614 have been developed to construct prosthetic heart valves that are compressed onto an expandable stent. This family of valve designs can be inserted through the wall of the left ventricle for subsequent stent expansion and placement into the ventricle's outflow tract as a replacement for a defective native valve. Unfortunately, due to design and manufacturing comprises required to compress this type of valve, the valve performance is considered sub-optimal to those knowledgeable in the art compared to non-compressible, currently used prosthetic valves. Currently, there are no devices to facilitate the placement of a non-compressible prosthetic valve through the wall of left ventricle without losing excessive blood or without applying excessive trauma to the valve implant. To those knowledgeable in the art of less invasive valve surgery, this invention described herein, when combine with the Cardiac Cannula Support device disclosed in U.S. Patent Application 20060247570 provide the necessary support devices and methods to enable the installation of a heart valve through the apex of the left ventricle.

A valve design such as that described in U.S. Pat. Nos. 7,081,089; 5,350,364; and 6,582,364 B2 may also be considered to insert a large device into an anatomical cavity without causing excessive trauma, but because these designs have only one sealing element, it would be very difficult to insert a device without loosing substantial body fluid.

Valve designs described by U.S. Pat. Nos. 5,041,095 and 5,782,817 represent a design style that could limit fluid loss, but because of the tight seals required to achieve this goal, the device being inserted is exposed to excessive trauma during insertion. Also, this class of design is best suited for smaller devices such as intravascular catheters. To those knowledgeable in medical device design, this type of design would not be useful to insert a large device such as a prosthetic heart valve.

### OBJECTS AND ADVANTAGES

The primary object of the anatomical cavity implant transport invention is to provide a device that can allow a doctor to safely insert relatively large devices into a body cavity without loosing significant amounts of fluid contained within the cavity and without damaging the device due to excessive trauma incurred by the device during insertion. A specific object of this invention is to allow a cardiac surgeon to insert a large diameter, mostly incompressible heart valve prosthesis through the wall of a beating heart without significant blood loss and without damage to the fragile device.

The invention has the following advantages:
The implant transport invention allows the insertion of a tool or implant into a body cavity without requiring the tool or implant to be inserted through a tight fitting seal.
The implant transport invention allows the insertion of a tool or implant into a body cavity without substantial fluid loss.
The implant transport invention provides for a minimally invasive insertion procedure since the inside passageway diameter of the invention is only slightly smaller than the outside diameter of the device.
The internal diameter of the implant transport invention need not be sized in close relationship to the size of the implant or tool being inserted to ensure an adequate fluid tight seal.
The anatomical cavity implant transport invention allows insertion of an implant while relying on near complete fluid isolation between the higher pressure anatomical cavity and the typically lower pressure ambient room environment thereby preventing excessive fluid loss.

These and other objects and advantages of this invention are achieved by an anatomical cavity implant transport invention comprising the following elements: three rigid hollow cylindrical elements aligned along a common axis, an elastomeric tubular element located within the housings, attachment means to connect the elastomeric tubular element to the three rigid hollow cylindrical elements, independent rotational means such as torsion springs to selectively bias the elastomeric tubular element into a twisted, closed configuration at each boundary between the three rigid hollow cylindrical elements.

The above mentioned objects and advantages of this invention will become apparent from the following description taken in connection with the accompanying drawings, wherein is set forth by way of illustration and example, preferred embodiments of this invention.

### DESCRIPTION OF DRAWING FIGURES

In the drawings, closely related figures have the same number but different alphabetic prefixes.

FIG. 1 A shows a perspective view of one embodiment of the invention.

FIG. 1B shows a side view of the device shown in FIG. 1A.

FIG. 1C shows a cross-section view of the device shown in FIG. 1B

FIGS. 2A-D shows an end view of an embodiment of the invention showing a sealing mechanism.

FIGS. 3A-C shows an end view of an embodiment of the invention showing a sealing mechanism around a convex shape.

FIG. 4A-M shows one embodiment of the invention in use.

FIG. 5A-B shows an end view and a cross-section view of a monolithic element used in one embodiment of the invention.

### DEFINITIONS

The terms “proximal” and “distal,” when used herein in relation to instruments used in the procedure of the present invention respectively refer to directions closer to and farther away from the operator performing the procedure.

### GENERAL SUMMARY OF INVENTION

This invention describes a device and method for inserting a large diameter object into a blood filled, pressurized cavity in the body without applying undue trauma to the object as it passes through the device and without loosing significant amounts of blood. More specifically, but not exclusively, this invention describes a device and method for inserting implants and related tools into a ventricle of a beating heart.

The anatomical cavity implant transport device invention comprises the following basic elements:
three rigid hollow cylindrical elements aligned along a common axis
an elastomeric tubular element located adjacent the hollow cylindrical elements
an attachment means to connect the elastomeric tubular element to the three rigid hollow cylindrical elements
independent rotational means such as torsion springs to selectively bias the elastomeric tubular element into a twisted, closed configuration at each boundary between the three rigid hollow cylindrical elements
a cavity access element to gain access into the anatomical cavity.

The invention can be further explained as follows. The three generally rigid hollow cylindrical elements or housings are aligned along a common axis and generally spaced a small distance apart. The proximal and distal housings can rotate about this common axis relative to the middle, or cargo housing. Adjacent the rigid housings is a cylindrical elastomeric member, an example being a thin-walled polyurethane tube. The tube is securely attached around its circumference to each rigid cylindrical housing.

If the proximal housing is rotated relative to the cargo housing, the polyurethane tube collapses in a twisting fashion along the circumferential boundary between the two housings. In a similar fashion, if the distal housing is rotated relative to the cargo housing, the polyurethane tube collapses in a twisting fashion along the circumferential boundary between these two adjoining housings. When the elastomeric tube is rotated at both boundaries, the interior space created between the twisted segments forms a cargo space suitable to transport a device safely into a body cavity. By sequentially opening and closing the two twisted segments of the tube, the implant, if sized to fit into the instantaneously formed cargo compartment, can be transported through the device without fluid loss and without traumatic insult. It should be noted that the circumferential width of the elastomeric tube to the cargo housing attachment defines the maximum width of the cargo space. The internal diameter of elastomeric tube defines the maximum diameter of cargo space.

This basic mechanism is interesting, but not directly useful as a medical device to control passage of a large object into an anatomical cavity since in its normal position, the passageway is open, allowing fluid to easily leave the anatomical cavity. To those knowledgeable in the art, it would be necessary for the device to be normally in the closed position, that is, with the elastomeric tube twisted closed at one or both twisting locations.

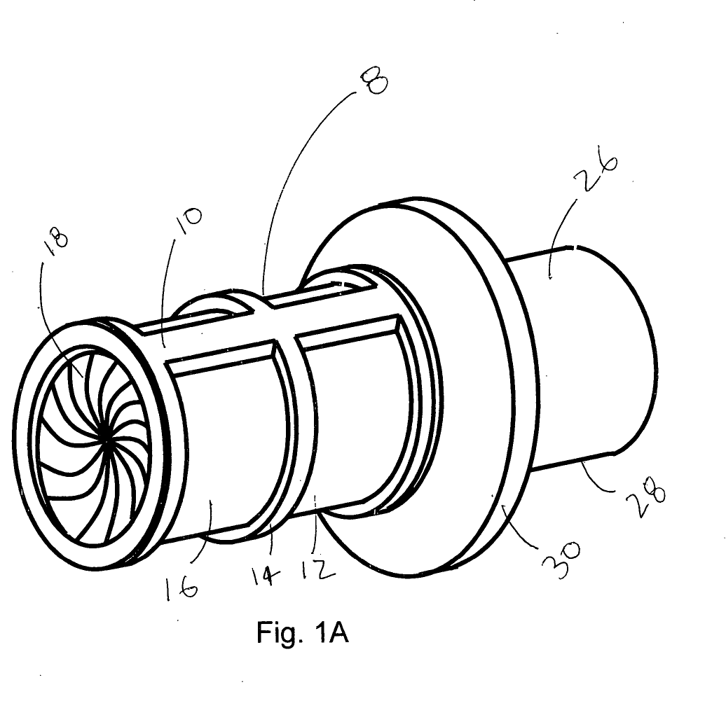

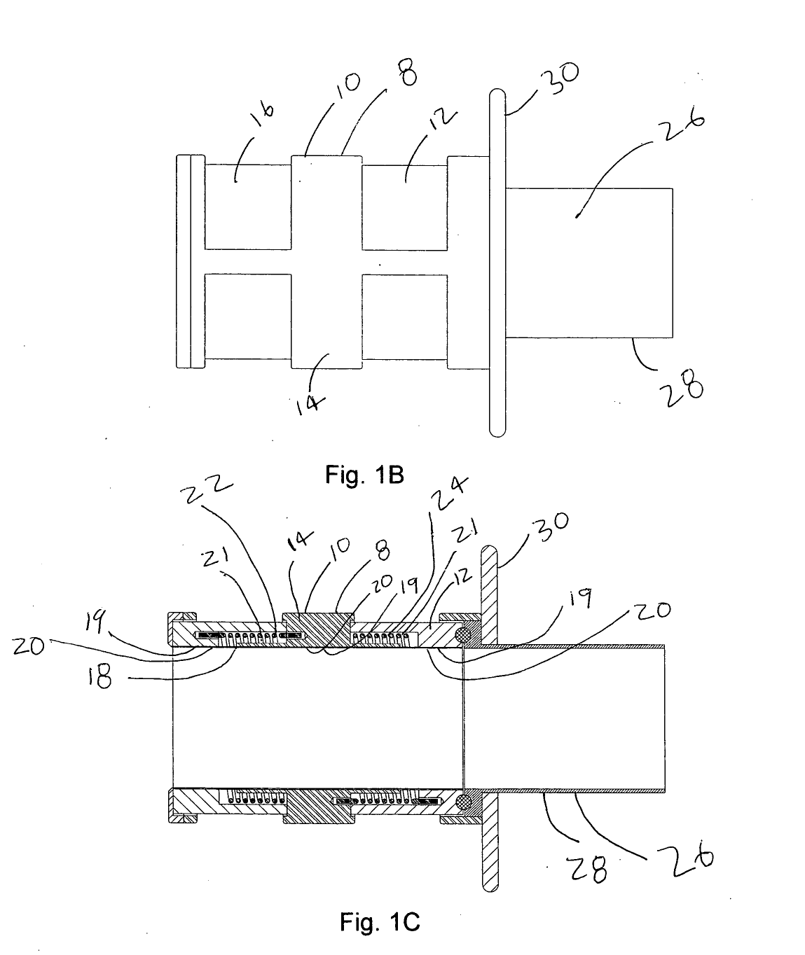

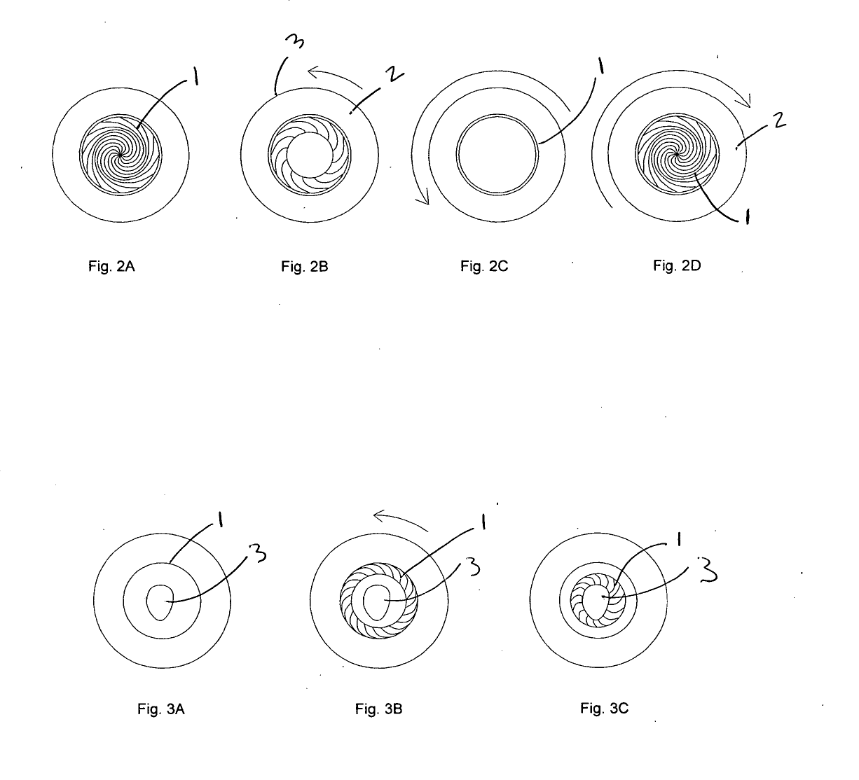

To ensure a normally closed condition, in one embodiment of this basic invention, two torsion springs are located around the rigid housings—one spring straddling the proximal and cargo housing; the other spring straddling the cargo and distal housings. It is well known to those in the art that a torsion spring can be twisted or biased to create a torque or twisting force. In this invention, specific torsion springs are selected so that each spring has a rotational force or torque stronger than the torque developed by the fully twisted elastomeric member. During assembly of the device, one end of one torsion spring is attached to the proximal housing. The housing is then rotated to twist the segment of the elastomeric member located between the proximal and cargo housings such that the elastomeric tube diameter at that location is near zero. When in this twisted position, the other end of the torsion spring is attached to the cargo housing. Since the force required to rotate the spring is stronger than the twisting force stored in the elastomeric tube, the elastomeric tube remains twisted closed with an opening diameter near zero. Another similar torsion spring is attached in a similar fashion between the cargo and proximal housings. It can be realized that in this normal, low energy state, the elastomeric tube is twisted at two locations, specifically, near the boundary between the proximal and distal housings and near the boundary between the cargo and distal housings. This design embodiment is shown in FIGS. 1A-C.

One torsion spring could be used to simultaneously bias both sections of the elastomeric tube by attaching the spring between the distal housing and the proximal housing.

If one applies the necessary counter force to rotate the proximal housing against the restraining force of the torsion spring, the spring force can be overcome and the elastomeric tube can be opened. Depending how far the housing is rotated, the elastomeric tube can be opened a controlled amount, ranging from barely open to fully open. This relative opening in the elastomeric tubular element is shown in FIG. 2A-D. In FIG. 2A, an Elastomeric Tube 1 is shown tightly closed, a condition considered “normal” since in this mode the torsion spring (not shown) has naturally overpowered the twisted tube's ability to open. In FIG. 2B, the user has overpowered the torsion spring by rotating a Housing 2 relative to a Cargo Housing 3 (hidden in this figure). In FIG. 2C, the user has continued to overpower the torsion spring until the Elastic Tube 1 is fully opened. In FIG. 2D, the user, moments before, has let go of the Housing 2 and the torsion spring has overpowered the Elastic Tube 1 to regain the normal closed mode creating a hemostatic seal.

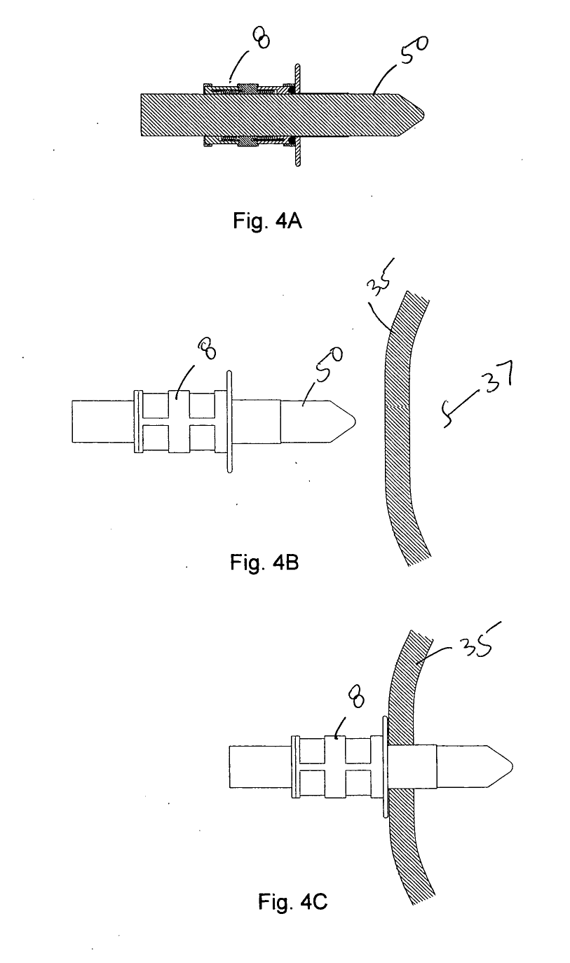

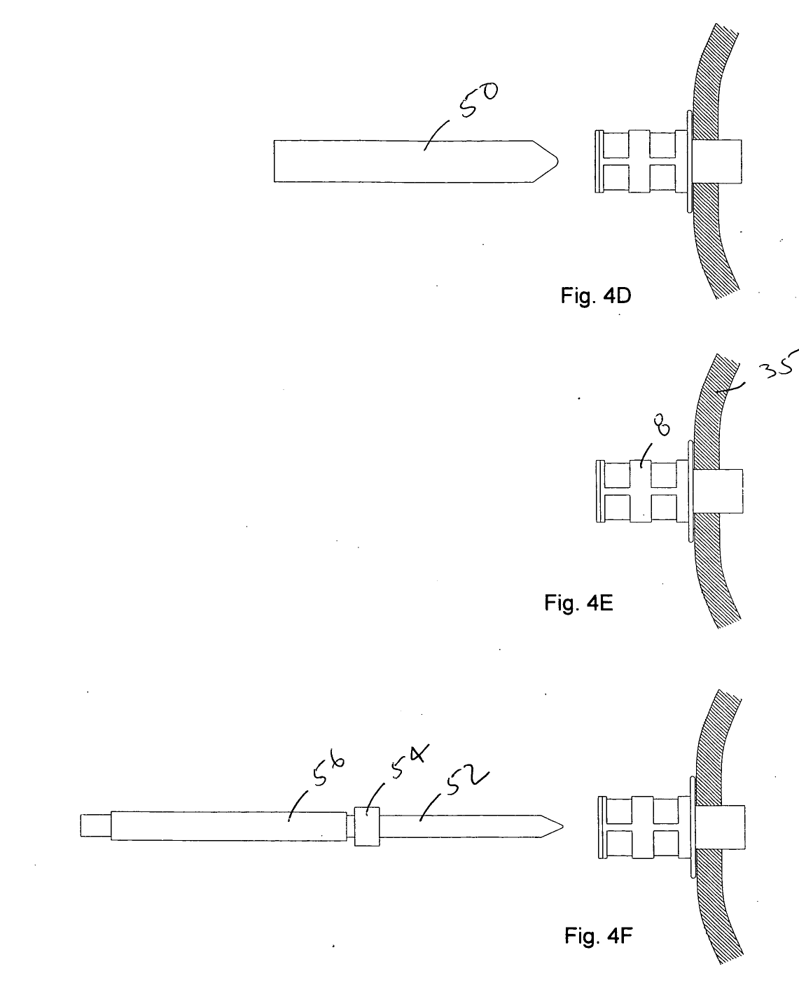

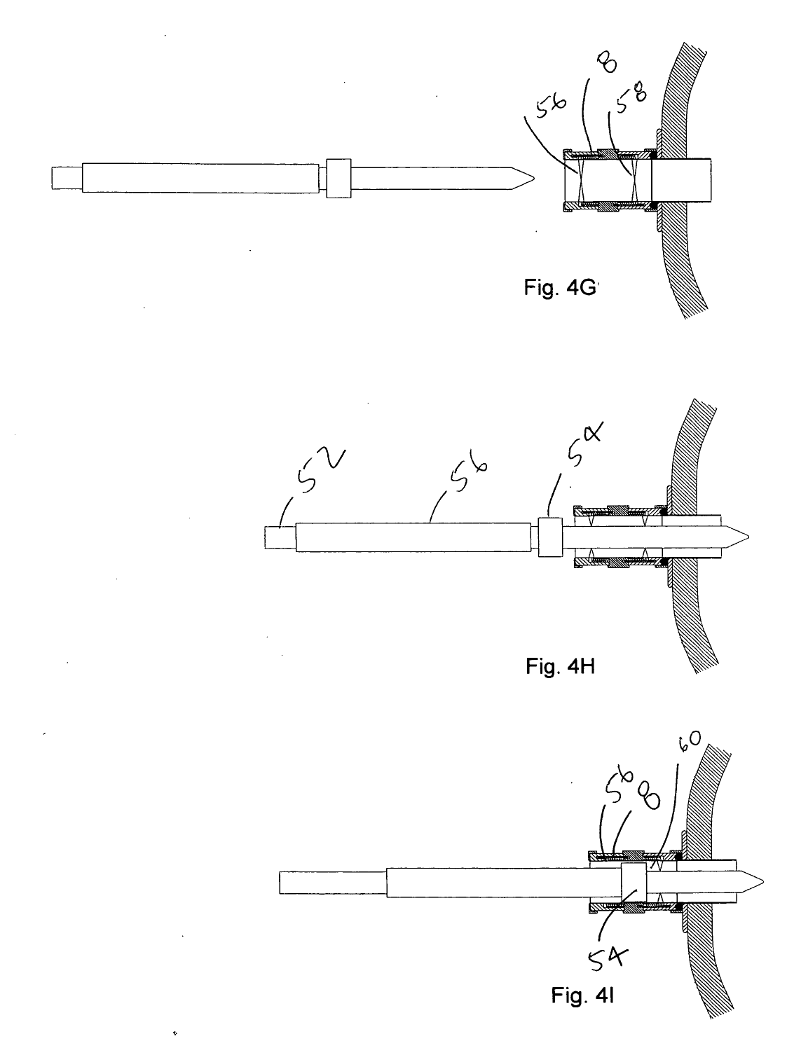

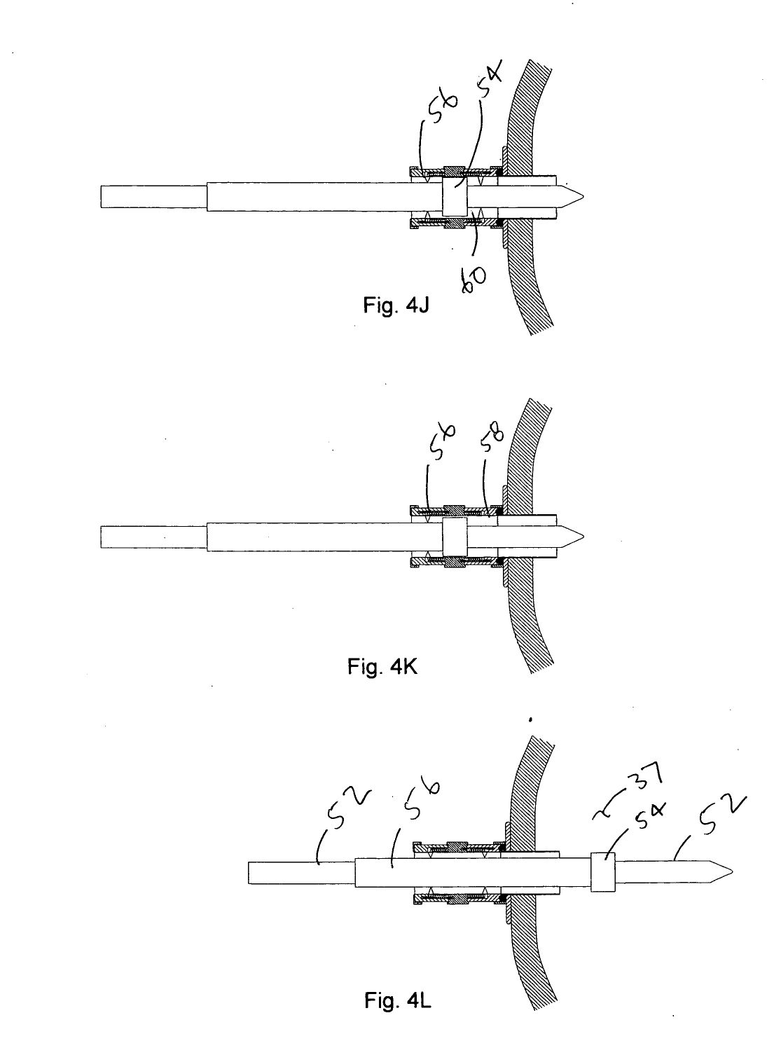

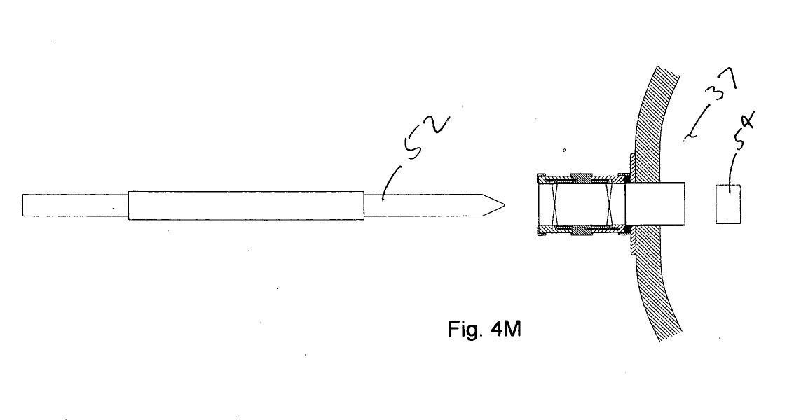

It should be clear to those knowledgeable in the art that a seal sufficient to stopping excessive fluid loss can be made by the twisted Elastomeric Tube 1 even if a Device 3 is present in the lumen as shown in FIG. 4. The Elastomeric Tube 1 will reduce in diameter until the seal twisting reaches force equilibrium with the torsion spring as shown in FIG. 3C. If an object is present, the seal will still be made. It can be further observed, that the invention could seal around objects having many different convex cross sectional shapes, not just circular shapes.

A cavity access element is located at the distal end of the device. It is designed to be inserted into a wall of the cavity. If necessary to ensure an adequate seal is maintained around the exterior of the access element, a cuff or other sealing element can be located around the periphery of the access element and position in closed contact with the exterior surface of the cavity. If necessary, the cuff could be bonded, sutured or otherwise temporarily affixed to the cavity surface.

### Description of Invention Structure

A preferred embodiment of the Anatomical Cavity Implant Transport Device 8 is shown in FIGS. 1A, 1B, and 1C. The Device 8 is comprised of the following elements: a Cylindrical Element 10, an Elastomeric Tubular Element 18, an Attachment Means 19 to connect the Elastomeric Tubular Element 18 to the Cylindrical Element 10 at three locations, Rotational Means 21 to selectively bias the Elastomeric Tubular Element 18 into a twisted, closed configuration at two locations.

This embodiment can be further described. The Cylindrical Element 10 is comprised of a three independent components; a Distal Housing 12, a Cargo Housing 14, and a Proximal Housing 16. These housings are composed of suitable rigid biomedical materials such as a plastic, like polycarbonate or polyester, or a metal such as stainless steel. A Cylindrical Elastomeric Member 18 is located adjacent to and coaxial to the Cylindrical Element 10. The Elastomeric Member 18 is composed of latex, silicone, polyurethane or some other suitable flexible biomaterial. An Adhesive 20 is used to create a circumferential attachment between the Distal Housing 12, the Cargo Housing 14, and the Proximal Housing 16 to the Cylindrical Elastomeric Member 18. Note the Elastomeric Member 18 is attachment to the entire internal surface of Cargo Housing 14.

The Proximal Torsion Spring 22, or some other rotation means, is located between and attached to the Proximal Housing 16 and the Cargo Housing 14. The Proximal Torsion Spring 22 is pre-twisted before attachments are made causing the Elastomeric Member 18 to be normally twisted and reduced in diameter to near zero at the circumferential boundary between the two housings. Only by applying an external force, such as that supplied by the fingers of a doctor, can the Proximal Housing 16 be rotated against the force of the Proximal Spring 22 to establish a fully circular shape of the Cylindrical Elastomeric Member 18. This shape is suitable to pass a large device just slightly smaller that the internal diameter of the fully open Elastomeric Member 18. When the external force is removed, the stored energy in the Distal Torsion Spring 24 causes the Cylindrical Elastomeric Member 18 to revert back to a twisted, closed shape that will not allow fluid to flow.

Intended to perform similar function as the Proximal Spring 22, the Distal Torsion Spring 24, or some other rotation means, is located between and attached to the Distal Housing 12 and the Cargo Housing 14 causing the Elastomeric Member 18 to be normally twisted and reduced in diameter to near zero at the circumferential boundary between the two housings. Again, only by applying an external force, such as that supplied by the fingers of a doctor, can the Proximal Housing 16 be rotated against the force of the Proximal Spring 22 to establish a circular shape of the Cylindrical Elastomeric Member 18. This shape is suitable to pass a large device. When the external force is removed, the stored energy in the Distal Torsion Spring 24 causes the Cylindrical Elastomeric Member 18 to revert back to a twisted, closed shape that does not allow fluid to flow.

The distal edge of the Cargo Housing 14 is bonded or otherwise attached to a Cavity Access Element 26. The Cavity Access Element 26 is composed of a thin walled rigid Tubular Element 28 sized in length to accommodate the thickness of the cavity wall intended to enter. Around the periphery of Tubular Element 28 is an Attachment Cuff 30. The Attachment Cuff 30 provides a surface to temporarily attach the Anatomical Cavity Implant Transport Device 8 to the wall of the Anatomical Cavity. Attachment is made using a temporary adhesive or by using temporarily placed sutures or by using some other temporary attachment means.

The diameters of the housings and elastomeric member are selected based on the anticipated diameter of the device or tool to be inserted through the device. The sizes of devices intended to be inserted through the invention could range from 1 mm to 27 mm in a heart procedure, 1 to 50 mm in a stomach procedure, and other ranges depending on the particular procedure.

### Operation

In FIG. 4a-4m is a schematic showing how an a large diameter object, such as a Heart Valve 54, could be passed thru a preferred embodiment of the Anatomical Cavity Implant Transport Device 8 into an Anatomical Cavity 35 without causing any trauma to the Valve 54 while maintaining a blood tight seal at all times.

FIG. 4A shows a cross section view of Device 8 with a Dilator 50 inserted through the device.

FIG. 4B shows Device 8 loaded with Dilator 50 ready for insertion through Cavity Wall 35 into Cavity 37.

FIG. 4C shows Device 8 inserted through Cavity Wall 35.

FIG. 4D shows the removal of Dilator 50. There is no fluid loss since both elastomeric seals are naturally biased to be closed.

FIG. 4E show Device 8 attached to the Cavity Wall 35.

FIG. 4F shows a Valve Delivery Tool 52 sized to carry the prosthetic Heart Valve 54. The Valve Delivery Tool 52 has a larger diameter Slideable Tube 56 that allows advancement of the Valve 54 on the Tool 52.

FIG. 4G shows Device 8 in cross-section revealing Proximal Elastomeric Seal 56 and the Distal Elastomeric Seal 58.

FIG. 4H shows the Valve Delivery Tool 52 inserted through both elastomeric seals. The seals have opened just sufficient to allow the Tool 52 to advance.

FIG. 4I shows the Valve 54 being advanced through a user opened Proximal Seal 56 into a Cargo Bay 60.

FIG. 4J shows the Proximal Seal 56 released by user to its normally closed position. The Valve 54 is fully enclosed in the Cargo Bay 60 defined by the elastomeric seals.

FIG. 4K shows the Distal Seal 58 opened by the user. Fluid from the cavity cannot exit the device due to the normally closed Proximal Seal 56.

FIG. 4L shows the Valve Delivery Tool 52 and associated Slideable Tube 56 advancing the Valve 54 fully into Cavity 37.

FIG. 4M shows the Valve 54 placed within Cavity 37 and Delivery Tool 52 removed without fluid loss due to the bias of the elastomeric seals to be in the closed position.

### SUMMARY, RAMIFICATIONS, AND SCOPE

In summary, the invention consists of the following basic elements:
three rigid hollow cylindrical elements aligned along a common axis
an elastomeric tubular element located adjacent the hollow cylindrical elements
an attachment means to connect the elastomeric tubular element to the three rigid hollow cylindrical elements
independent rotational means such as torsion springs to selectively bias the elastomeric tubular element into a twisted, closed configuration at each boundary between the three rigid hollow cylindrical elements
a cavity access element to gain access into the anatomical cavity.

When compared to prior art, the invention by its unique design has significant advantages as described below:
Because the elastomeric tube diameter can be user adjusted to any size between nearly closed and completely open, the invention allows the insertion of a tool or implant into a body cavity without requiring the tool or implant to be inserted through a tight fitting seal
Because the elastomeric tube can be closed tight at two locations, an implant can be sequentially moved through the device with one valve always in the closed condition, thereby allowing the insertion of a tool or implant into a body cavity without substantial fluid loss
The invention provides for a minimally invasive insertion procedure since the inside passageway diameter of the invention is only slightly smaller than the outside diameter of the cavity access element of the device
Because the internal diameter of the implant transport invention need not be sized in close relationship to the size of the implant or tool being inserted to ensure an adequate fluid tight seal., the implant or device is not submitted to excessive trauma or friction during insertion through the cavity wall
Because the elastomeric tube can be closed tight at two locations, the anatomical cavity implant transport invention allows for sequential insertion of an implant while relying on near complete fluid isolation between the higher pressure anatomical cavity with the typically lower pressure ambient room pressure thereby preventing excessive fluid loss

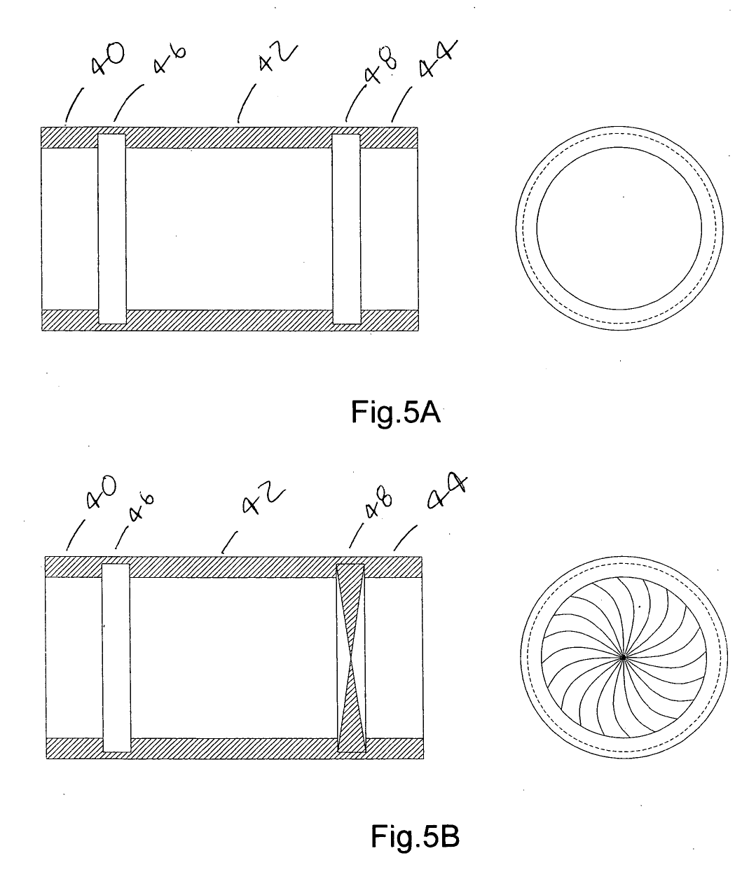

Although the description above contains many specifications, these should not be construed as limiting the scope of the invention but as merely providing illustrations of the presently preferred embodiment of this invention. For example:
The volume of the cargo space (length and diameter) between the proximal and distal twisted segments of the elastomeric member can be varied to accommodate the anticipated size of the implant intended to be transported into the heart.
One valve could be removed from the design. In some situations, for instance a catheter only insertion, only one valve could provide a sufficient level of hemostasis.
A venting element could be installed within the device to vent any gas captured within the device during operation. This would be important if the procedure required that no outside gas enter the anatomical cavity during the procedure.
At the end of the procedure, an implantable plug could be inserted through the device. When the device is removed, the implantable plug would be left in the cavity wall. The plug could then be sutured or otherwise permanently attached to the cavity wall.
The cylindrical element and the elastomeric tubular element could be combined into one element. This element is shown in FIG. 5A-B. In this design, both the cylindrical housing element and the elastomeric tube elements are incorporated into one monolithic cylinder having more rigid annular segments defined as Rigid Proximal Segment 40, Rigid Cargo Segment 42, and Rigid Distal Segment 44 adjacent to less rigid annular segments Flexible Proximal Segment 46 and Flexible Distal Segment 48. If a rotational force is applied between two adjacent rigid segments as shown in FIG. 5B, the intervening flexible annular segment will collapse and twist closed, The other invention elements previously described could be incorporated with this monolithic element to create one embodiment of the invention.

Thus, the scope of the invention should be determined by the appended claims and their legal equivalents rather than by the examples given.

1. An anatomical cavity implant transport and sealing assembly, comprising:
a) a cylindrical housing member, said housing member comprising a distal section, a cargo section, and a proximal section aligned along a common axis;
b) a cylindrical elastomeric member, said cylindrical elastomeric member located adjacent to and coaxial with said cylindrical housing member;
c) attachment means, said attachment means capable of creating a circumferential attachment of said distal section, said cargo section, and said proximal section of said cylindrical housing member to said cylindrical elastomeric member;
d) a rotation means, said rotation means capable of independently rotating said proximal section and said distal section of said cylindrical housing member relative to said cargo section of said cylindrical housing member causing said elastomeric member to reduce in diameter at two locations spaced along the axis; and
e) a cavity access element.

2. The anatomical cavity implant transport and sealing assembly of claim 1 wherein the cylindrical elastomeric member is composed of polyurethane.

3. The anatomical cavity implant transport and sealing assembly of claim 1 wherein the attachment means is an adhesive.

4. The anatomical cavity implant transport and sealing assembly of claim 1 wherein the rotation means is a torsion spring.

5. The anatomical cavity implant transport and sealing assembly of claim 1 wherein the cylindrical housing member and the cylindrical elastomeric member are merged into one monolithic element.

6. The anatomical cavity implant transport and sealing assembly of claim 1 wherein the cavity access element has an internal diameter of between 5 mm and 30 mm.
